(19) United States
(12) Patent Application Publication
(10) Pub. No.: US 2008/0097509 A1
(43) Pub. Date: Apr. 24, 2008

(54) Biodegradable Closure Device

(76) Inventors: Mordechay Beyar, Caesarea (IL); Oren Globerman, Kfar-Shemaryahu (IL); Amir Loshakove, Moshav Bazra (IL); Eran Goldberg, Nesher (IL); Ido J. Kilemnik, Herzlia (IL)

Correspondence Address: PRTSI, P.O. Box 16446, Arlington, VA, 22215, US

(21) Appl. No.: 10/596,496

(22) PCT Filed: Dec. 15, 2004
(86) PCT No.: PCT/IL04/01134

§ 371 Date: Aug. 24, 2007

Related U.S. Application Data
(60) Provisional application No. 60/529,092, filed on Dec. 15, 2003.

(51) Int. Cl. A61M 29/00

(52) U.S. Cl. 606/192; 606/191

(57) ABSTRACT

A biodegradable balloon adapted to exert pressure on a hole formed in a lumen in the body when placed adjacent to the hole, inside the body, and expanded, and adapted to remain in place thereafter and to be absorbed by the body.

### RELATED APPLICATIONS

The present application claims the benefit under 119 (e) of U.S. Provisional Application No. 60/529,092 filed on Dec. 15, 2003, the disclosure of which is incorporated herein by reference.

### FIELD OF THE INVENTION

The field of the invention is devices which exert pressure on tissue inside the body, in particular to stop bleeding from holes in blood vessels.

### BACKGROUND OF THE INVENTION

Percutaneous transluminal coronary angioplasty and other medical interventions involve access into the vascular system. In many cases, the treatment or diagnostic device, such as catheter and balloon angioplasty device, is introduced into the vascular system via a catheter introducer/sheath, cannula or the like, which occasionally are of relatively large diameter to enable the passage of the treatment/diagnostic device through them. At the end of the intravascular procedure, following removal of catheter sheath/cannula, the blood vessel aperture should be closed and sealed, to stop/prevent bleeding.

During percutaneous transluminal coronary angioplasty, for instance, the catheter is normally inserted into the femoral artery, near the groin, through a introducer sheath having an internal diameter of about 5-9 French. Various means are currently in use in order to perform hemostasis to the femoral artery following arterial catheterization, including closure devices and constant manual pressure. The latter includes applying of direct pressure on the artery aperture site by a trained medical person for a period of twenty minutes or more. This method has a few drawbacks, as it is time consuming, poses a risk of hematoma and risk of reduction in blood flow due to the manual pressure, and requires hours without motion of the involved patient limb after the pressure is removed.

Among the closure devices, there are several devices in clinical use, such as the Angioseal, by Sherwood Medical International, USA, described in U.S. Pat. No. 6,179,863. This haemostatic puncture closure device comprises an anchor, a collagen sponge and a suture, all of which are biodegradable. The two former components are connected to the suture, while the anchor is located inside the artery and the collagen unit is incorporated into the hole in the wall of the artery, to achieve hemostasis.

PCT Applications PCT/IL99/00285, PCT/IL99/00674, and PCT/IB00/00302 describe an implantable nitinol closure device, which is loaded on a cannula or introducer sheath and penetrates the outer surface of the of the artery wall. Upon removal of cannula/introducer sheath the implant is deployed, resulting in closure of the artery puncture.

Another closure device is the Prostar Percutaneous Vascular Surgical Device, by Perclose Inc, USA, described in U.S. Pat. Nos. 5,902,311, 5,921,994, and 6,036,699. This closure device enables direct suturing of the puncture site of the artery. U.S. Pat. No. 6,117,145, also assigned to Perclose, describes a non-compliant hemostasis device that temporarily is pressed against the puncture from the outside of the blood vessel. U.S. Pat. No. 6,743,195 to Zucker, assigned to Cardiodex, as well as U.S. Pat. Nos. 5,728,134 and 6,048,358 to Barak, describe devices in which an inflatable anchor balloon is deployed inside an artery from which a catheter has been removed, and the anchor balloon is retracted until it engages the inner wall of the artery. Another balloon is placed just outside the artery by the catheter introducer, and expanded. The anchor balloon is then withdrawn from the artery, while the other balloon presses against the outside of the artery, preventing bleeding. However, since the other balloon is also withdrawn after a relatively short time, once bleeding stops, there is a danger that bleeding will resume again.

U.S. Pat. Nos. 4,852,568 and 4,890,612, to Kensey, describe a biodegradable device which is deployed inside a blood vessel, and then pulled tight against a puncture in the wall of the blood vessel, remaining there and eventually being absorbed into the body. Because these devices are located inside the blood vessel, there is a possibility that they will adversely affect blood flow.

### SUMMARY OF THE INVENTION

An aspect of some embodiments of the invention concerns a biodegradable balloon which is expanded inside the body, exerting pressure on body tissue. In some embodiments of the invention, the balloon exerts pressure on the wall of a blood vessel which has a hole in it, such as a puncture made for catheterization. The pressure exerted by the balloon substantially prevents bleeding from the hole, and the balloon remains in place and eventually disintegrates and is absorbed in the body. Because the balloon continues to press against the hole for an extended time, until it starts to disintegrate, there is less chance that the hole will start to bleed again, than if the balloon were not biodegradable and had to be withdrawn from the body after a short time. Optionally, the balloon is deployed on the outside of the blood vessel, which has the potential advantage that it does not interfere with blood flow.

Optionally a biodegradable anchor, inside the blood vessel, positions the balloon on the outside of the blood vessel, adjacent to the outer wall, and may also help to hold it in place there. Optionally, the balloon is deployed through a catheter introducer that was used for the catheterization. Optionally, the balloon is topologically a torus, and a guide wire runs through it, to help place the balloon precisely. Optionally, material used to expand the balloon is brought through the catheter introducer, and various mechanisms are optionally used to seal the balloon after it expands. In an alternative embodiment, the balloon is seal-sealing and the guide wire punctures a hole in the balloon. Upon removal of the guide wire, the puncture self-seals.

Alternatively, instead of being used to seal a hole in a blood vessel wall, the balloon is used to seal an hole in a hollow organ of the body, for example in the digestive system, respiratory system or urinary system, where the hole was made, for example, as part of a diagnostic and/or therapeutic medical procedure.

In other embodiments of the invention, the balloon exerts pressure on the outside of the urethra, to reduce its diameter and prevent or treat urinary incontinence, or the balloon is injected and expanded percutaneously, in order to treat wrinkles.

An aspect of an embodiment of the invention concerns a biodegradable leaf valve, located in a neck of the balloon. The valve allows the balloon to be filled with material through a filling tube that is connected to the neck, to expand the balloon, but the valve seals the balloon once it is expanded and removed from the filling tube.

An aspect of an embodiment of the invention concerns a method of removing the balloon from the filling tube, once the balloon is fully expanded. The neck goes around the outside of the end of the filling tube, when the filling tube is connected to the balloon. A relatively rigid pushing tube, closely fitting around the outside of the filling tube, is pushed down the filling tube until it reaches the neck of the balloon, and then pushes against the neck of the balloon while the filling tube is pulled out of the neck of the balloon.

An aspect of an embodiment of the invention concerns a method of positioning a biodegradable balloon on the outside of a blood vessel, or another lumen in the body, to seal an opening. The balloon is inserted into the blood vessel before inflating it, and is withdrawn until another element, which is attached to the distal end of the balloon and has been oriented so that it cannot fit through the opening, reaches the inner wall of the blood vessel. The balloon will then be located right outside the wall of the blood vessel, and is inflated there to seal the opening. Optionally, the element attached to the balloon acts as an anchor, and presses against the blood vessel wall from the inside, further helping to seal the opening.

There is thus provide din accordance with an exemplary embodiment of the invention, a biodegradable balloon adapted to exert pressure on a hole formed in a lumen in the body when placed adjacent to the hole, inside the body, and expanded, and adapted to remain in place thereafter and to be absorbed by the body. Optionally, the balloon requires between 1 and 2 days to be absorbed into the body, when placed on the outside of a blood vessel. Optionally, the balloon requires between 2 days and 1 week to be absorbed into the body, when placed on the outside of a blood vessel. Optionally, the balloon requires between 1 week and 2 weeks to be absorbed into the body, when placed on the outside of a blood vessel. Optionally, the balloon requires more than 2 weeks to be absorbed into the body, when placed on the outside of a blood vessel.

Optionally, the balloon is adapted to exert enough pressure to substantially stop bleeding from the hole, when the lumen is a blood vessel.

Optionally, the hole is a catheterization puncture in the blood vessel. Optionally, the blood vessel is an artery.

Alternatively or additionally, said balloon is inflated to a pressure of at least 1 bar. Alternatively or additionally, said balloon is inflated to a pressure of at most 6 bar. Alternatively or additionally, said balloon is elastically deformable when it expands.

In an exemplary embodiment of the invention, said balloon plastically deforms when it expands. Alternatively or additionally, the balloon comprises a channel for a guide wire.

Alternatively or additionally, the balloon comprises a sealing mechanism. Optionally, said sealing mechanism comprises a valve. Alternatively or additionally, said sealing mechanism comprises a self-adhesive channel. Alternatively or additionally, said sealing mechanism comprises a self-sealing channel. Alternatively or additionally, said sealing mechanism comprises a knotted channel.

In an exemplary embodiment of the invention, the balloon is coated on an outside surface thereof with an adhesive material.

In an exemplary embodiment of the invention, the balloon is coated on an outside surface thereof with an anti-adhesive material.

In an exemplary embodiment of the invention, the balloon is coated on an inside surface thereof with an anti-adhesive material.

There is also provided in accordance with an exemplary embodiment of the invention, a balloon system comprising a balloon as described above and also comprising a biodegradable anchor element coupled to said balloon and adapted to remain in a blood vessel on adjacent said hole.

There is also provided in accordance with an exemplary embodiment of the invention a system for hemostasis of a hole in a blood vessel, the system comprising:
a) a biodegradable balloon;
b) a delivery system capable of placing the balloon adjacent to the hole; and
c) a filling tube through which a filling material passes to expand the balloon.
   Optionally, the system comprises a reservoir of biodegradable filling material. Alternatively or additionally, the system comprises a pusher adapted to separate said filling tube from said balloon. Alternatively or additionally, said balloon is adapted to remain outside of a blood vessel while sealing said blood vessel. Alternatively or additionally, the system comprises a guide wire adapted to guide said balloon.

There is also provided in accordance with an exemplary embodiment of the invention a biodegradable check valve adapted to seal an inflatable biodegradable balloon implanted inside the body. Optionally, said valve is formed of a same material as said balloon. Alternatively or additionally, said valve is adapted to withstand a pressure of at least 1 bar of a liquid without leaking. Alternatively or additionally, said valve has a diameter of less than 3 mm.

In an exemplary embodiment of the invention, said valve is a leaf valve. Optionally, said leaves have a thickness of less than 2% of said diameter.

There is also provided in accordance with an exemplary embodiment of the invention, a method of sealing an opening in a hollow structure in the body, the method comprising:
a) positioning an uninflated biodegradable balloon outside the structure, adjacent to the opening;
b) inflating the balloon, causing the balloon to press against the opening, at least partially sealing it;
c) leaving the balloon in place until it degrades and is absorbed by the body;
   wherein the balloon does not degrade sufficiently to stop pressing against the opening until after the opening seals. Optionally, positioning comprises positioning using an introducer sheath. Alternatively or additionally, the method comprises using a same sheath for positioning as for introduction of a tool into said hollow structure. Alternatively or additionally, positioning comprises positioning using a biodegradable anchor element attached to said balloon. Optionally, inflating comprises engaging said hollow structure between said anchor and said balloon.

In an exemplary embodiment of the invention, positioning comprises positioning using a guide wire. Alternatively or additionally, inflating comprises inflating with a curable material.

In an exemplary embodiment of the invention, inflating comprises inflating with a non-curable material. Optionally, inflating comprises sealing.

In an exemplary embodiment of the invention, leaving comprises pushing said balloon off of a filling tube.

There is also provided in accordance with an exemplary embodiment of the invention, a method of manufacturing a biodegradable check valve adapted to seal an inflatable biodegradable balloon implanted inside the body, the method comprising:
a) plating a first portion of a rod with a first portion of a biodegradable material;
b) plating a second portion of the rod with a second portion of the biodegradable material that is thinner than the first portion of the biodegradable material;
c) removing the plated material from the rod without tearing the plated material; and
d) crimping the second portion of the biodegradable material, while applying sufficient heat to said second portion so that said material undergoes plastic deformation, thereby forming leaves of a leaf valve.

There is also provided in accordance with an exemplary embodiment of the invention, a method of implanting an inflated balloon inside the body, the method comprising:
a) providing a balloon having a neck thereof mounted around a distal end of a filling tube;
b) placing the balloon inside the body while the neck is around the distal end of the filling tube and a more proximal portion of the filling tube remains outside the body;
c) inflating the balloon through the filling tube;
d) applying a pushing force against said neck; and
e) leaving the inflated balloon inside the body.

There is also provided in accordance with an exemplary embodiment of the invention, a system for hemostasis of a hole in a blood vessel, comprising:
a) a biodegradable expandable element; and
b) a biodegradable anchoring element attached to the expandable element; wherein, when the expandable element is expanded and located adjacent to the hole outside the blood vessel, and the anchoring element is located adjacent to the hole inside the blood vessel, the expandable element is capable of exerting sufficient pressure on the hole to achieve hemostasis.

### BRIEF DESCRIPTION OF THE DRAWINGS

Exemplary embodiments of the invention are described in the following sections with reference to the drawings. The drawings are generally not to scale and the same or similar reference numbers are used for the same or related features on different drawings.

FIG. 1A is a side view of a system for hemostasis of a hole in a blood vessel, using a balloon and anchor, according to an exemplary embodiment of the invention;

FIG. 1B is a more detailed view of the balloon and anchor in FIG. 1A;

FIG. 2 is a side-view of an introducer sheath for catheterization of a blood vessel, according to the prior art;

FIG. 3 is a side view showing the hemostasis system of FIG. 1A inserted into a blood vessel through the introducer sheath of FIG. 2, according to an exemplary embodiment of the invention;

FIGS. 4A and 4B are a time sequence of side views of the anchor shown in FIG. 1B, showing how it is rotated before inserting the hemostasis system into the introducer sheath in FIG. 2;

FIGS. 5A to 5C, together with FIG. 3, form a time sequence of side views, showing how the hemostasis system shown in FIG. 1A is deployed on a blood vessel, through the introducer sheath shown in FIG. 2;

FIGS. 6A and 6B are a time sequence of side views showing a balloon being twisted to seal it after expanding, according to another exemplary embodiment of the invention;

FIGS. 7A to 7C are a time sequence of side views showing a balloon being filled through a loosely knotted tube which is then pulled tight to seal the balloon, according to another exemplary embodiment of the invention;

FIGS. 8A to 8D are a time sequence of side views showing a balloon being filled through a self-sealing puncture, according to another exemplary embodiment of the invention;

FIG. 9A and FIG. 9B are side and axial views, respectively, of a balloon with a guide wire going through it, according to another exemplary embodiment of the invention;

FIG. 10 is a side view of a system for hemostasis of a hole in a blood vessel, according to the embodiment of the invention shown in FIGS. 9A and 9B;

FIG. 11 is a side view of an introducer sheath for catheterization of a blood vessel, with a guide wire going through it, according to the prior art;

FIGS. 12A to 12E are a time sequence of side views showing how the hemostasis system shown in FIG. 10 is deployed on a blood vessel, through the introducer sheath shown in FIG. 11;

FIGS. 13A-13D are perspective views showing a method of manufacturing a biodegradable valve, according to an exemplary embodiment of the invention;

FIGS. 13E and 13F are side views of the valve shown in FIG. 13D;

FIGS. 13G and 13H are side cross-sectional views showing the method of operation of the valve shown in FIG. 13D; and

FIG. 14 is a side cross-sectional view showing a method of removing a balloon from a filling tube, according to an exemplary embodiment of the invention.

### DETAILED DESCRIPTION OF EXEMPLARY EMBODIMENTS

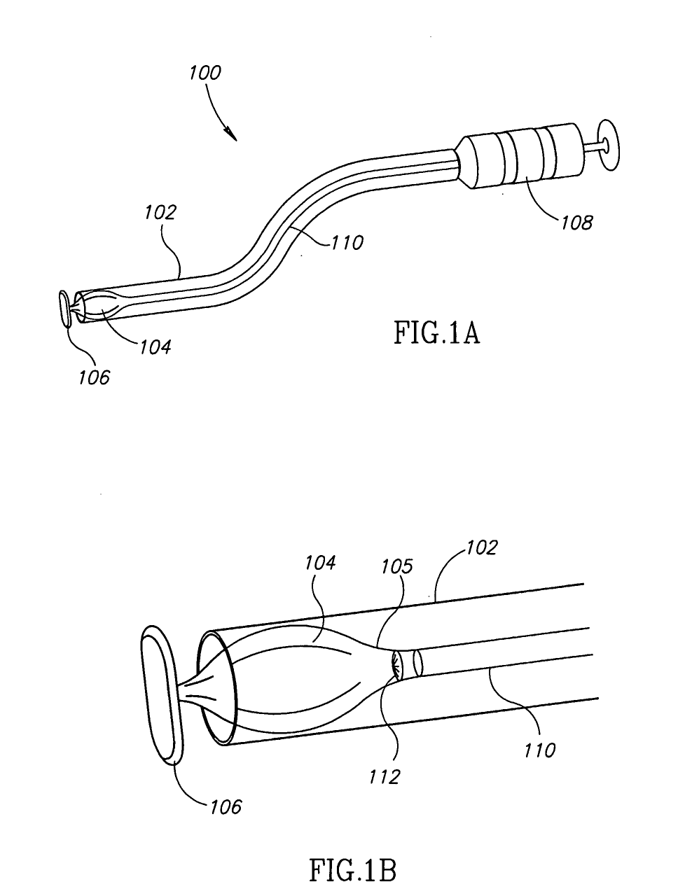

FIG. 1A shows a system 100 for hemostasis of a hole in a blood vessel wall. A flexible tube 102 has a balloon 104 inside it near the distal end, and there is a rod-shaped anchor 106, just past the distal end of flexible tube 102, attached to balloon 104. Balloon 104 is shown in a collapsed state. A syringe 108 filled with saline solution is located on the proximal end of tube 102, and is used to expand the balloon through a filling tube 110, which runs through tube 102.

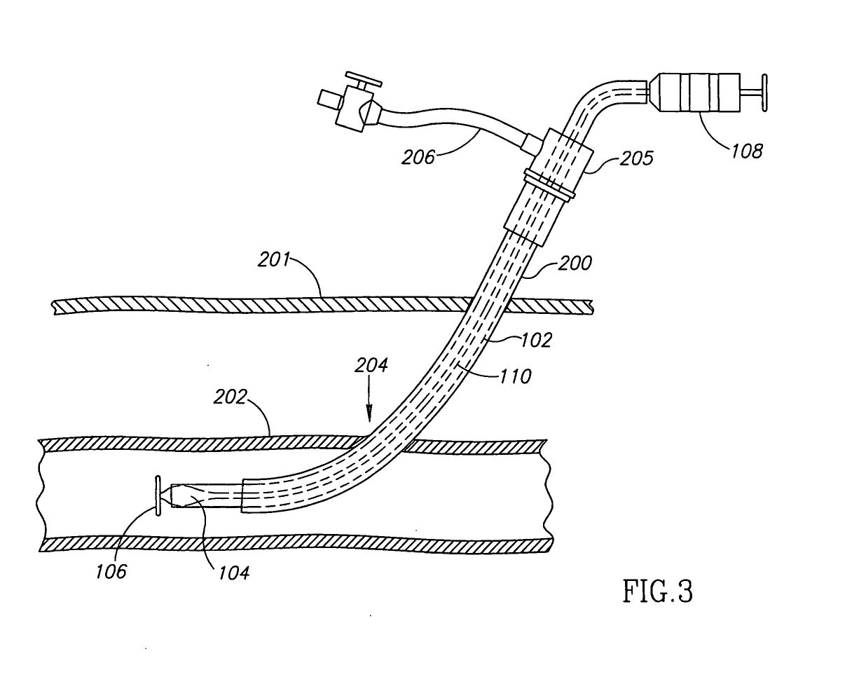

Alternatively, there is no tube 102, and system 100 consists of balloon 104, anchor 106, filling tube 110, and syringe 108. Having a tube 102 around the filling tube and balloon has the potential advantage of protecting these parts, and making it easier to place them into a blood vessel as will be described below in FIG. 3, for example by providing greater stiffness.

Alternatively, instead of balloon 104, there is an expandable biodegradable sponge.

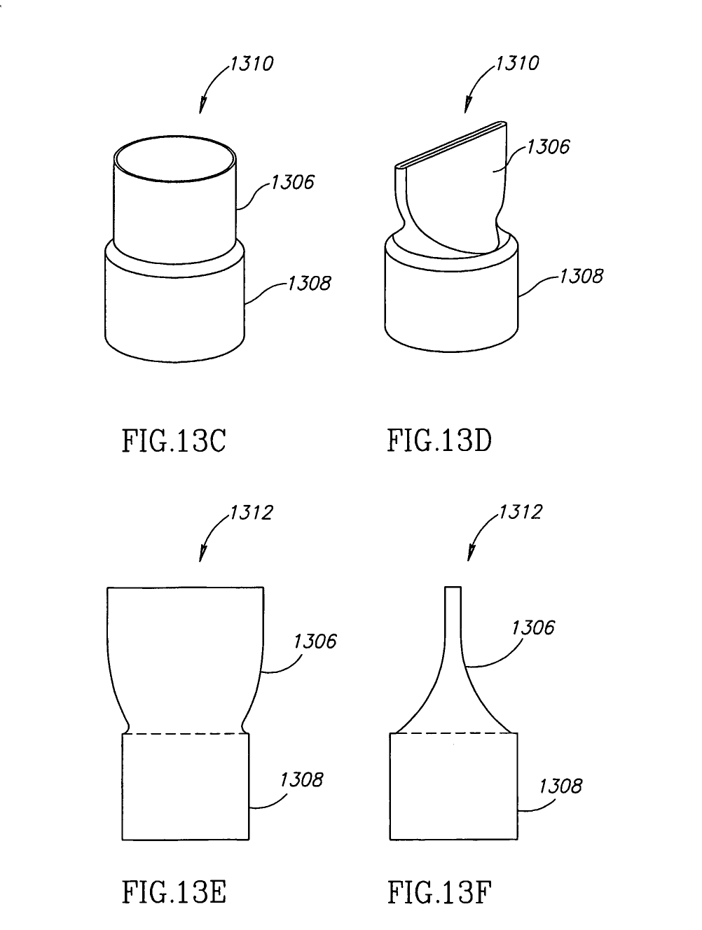

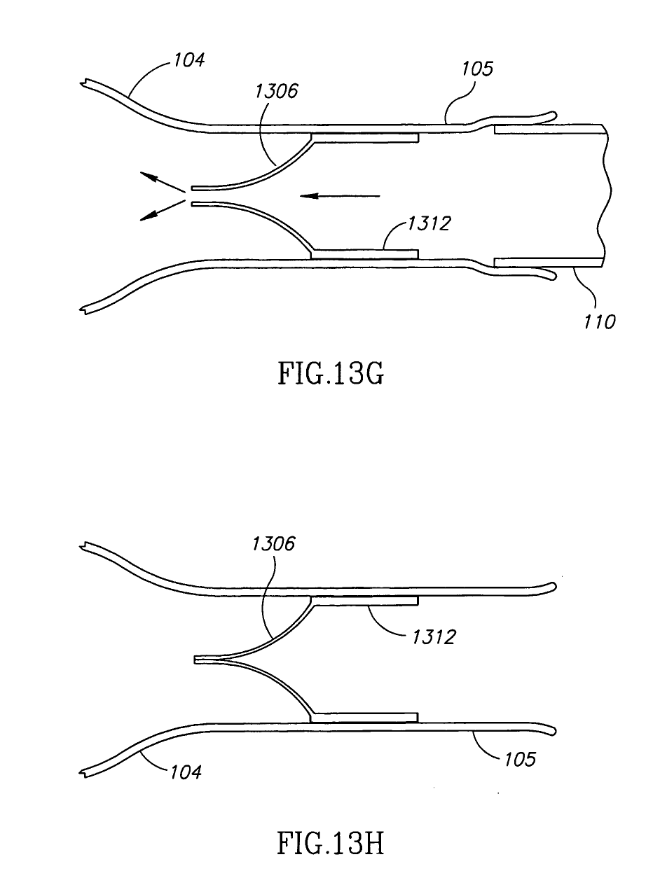

FIG. 1B is a more detailed view of the distal end of tube 102, including balloon 104 and anchor 106. Filling tube 10, inside tube 102, is connected at its distal end to a neck 105 of balloon 104, and at its proximal end to syringe 108, not shown in FIG. 1B. The saline solution flows through filling tube 110 into balloon 104, when balloon 104 is expanded. A check valve 112 in neck 105 optionally prevents the saline solution from flowing back out of balloon 104, after it is filled. Details about the design of check valve 112, and methods of manufacturing it, are described below in connection with FIGS. 13A to 13H.

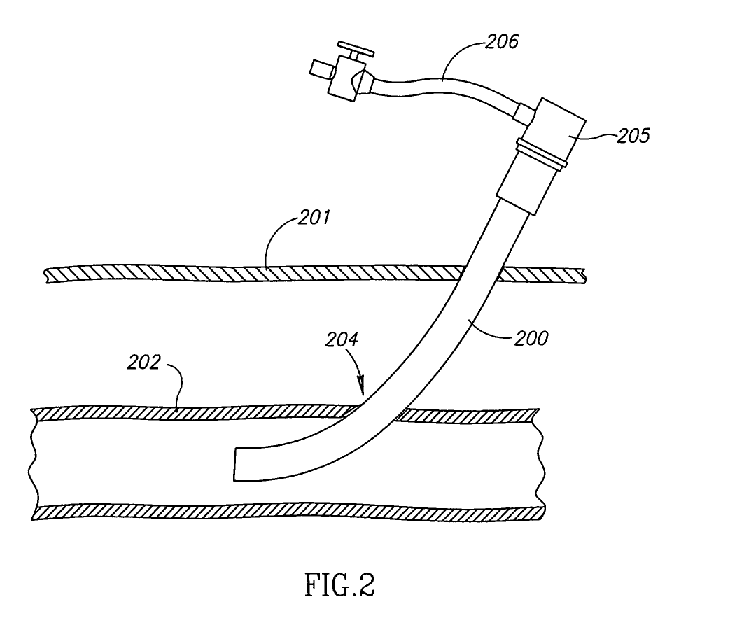

FIG. 2 shows an introducer sheath 200 inserted through a patient's skin 201 into a blood vessel 202, according to the prior art. Introducer sheath has previously been used to create a hole 204 in the wall of blood vessel 202, allowing the distal end of introducer sheath 200 to enter blood vessel 202. The introducer sheath is used to introduce a catheter (not shown in the drawings) into the blood vessel, through a port 205. A side lumen 206 is optionally used, for example, to take samples of blood, and to determine whether or not the distal end of the introducer sheath is in a blood vessel.

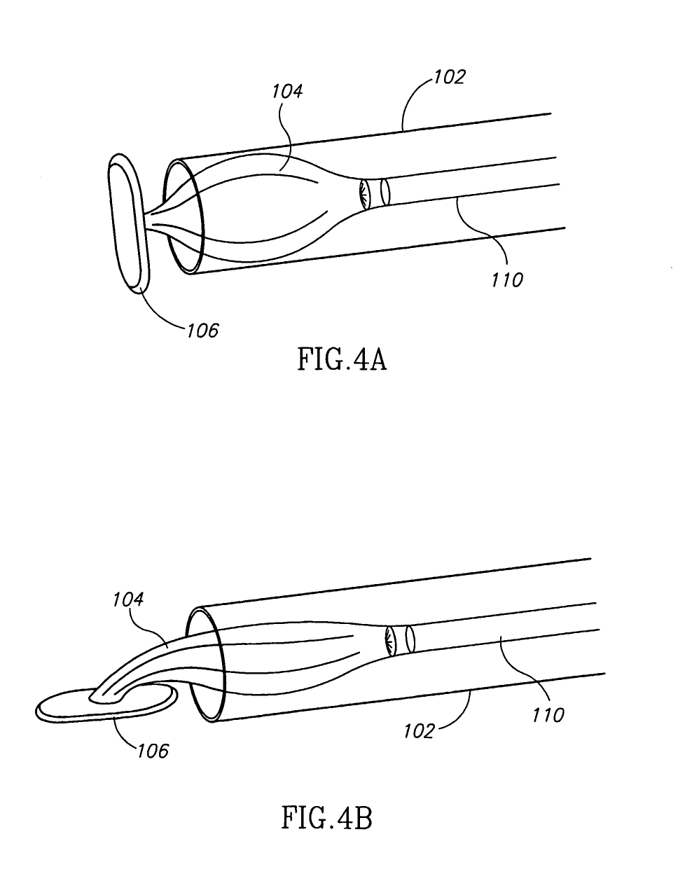

Once the catheterization procedure has been completed, and the catheter has been removed from introducer sheath 200, the sheath is used to introduce hemostasis system 100 into the blood vessel, as shown in FIG. 3. In FIG. 3, the distal end of tube 102, including balloon 104 and anchor 106, has passed through port 205 and sheath 200 into blood vessel 202. Anchor 106 may be too wide to fit through sheath 200 when it is oriented as shown in FIGS. 1A, 1B, and 3. Before inserting system 100 into sheath 200, anchor 106 is optionally rotated from its orientation in FIG. 4A to its orientation in FIG. 4B, which allows anchor 106 to fit through sheath 200. As may be seen in FIG. 4B, in order to rotate anchor 106 it is pulled a short distance out from the distal end of tube 102, together with part of balloon 104. Because balloon 104 is flexible in its deflated state, anchor 106 can now be rotated so that its longest dimension is parallel to the axis of tube 102. Once anchor 106 has been inserted into sheath 200, the sides of sheath 200 keep anchor 106 in that orientation until anchor 106 emerges from the distal end of sheath 200.

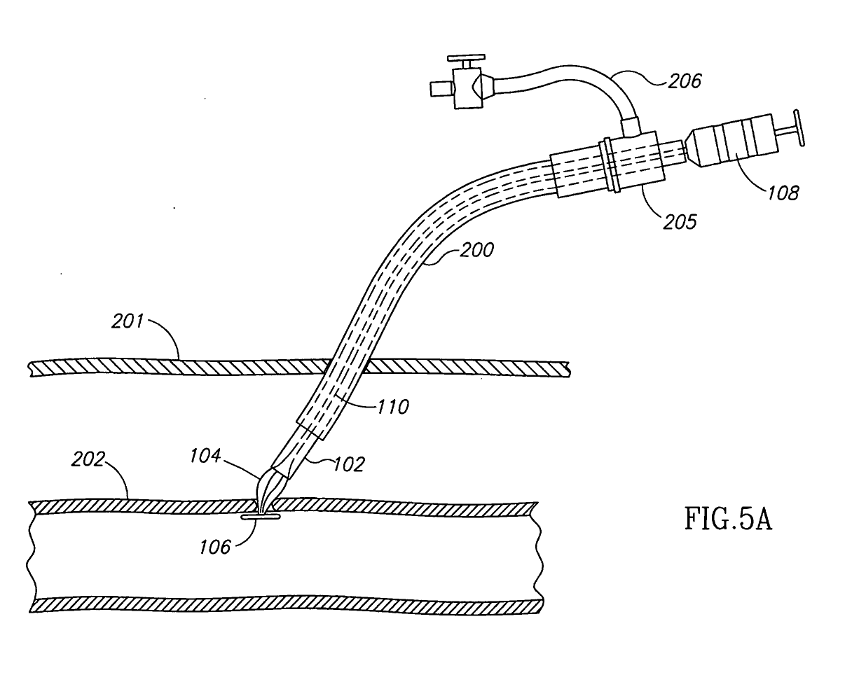

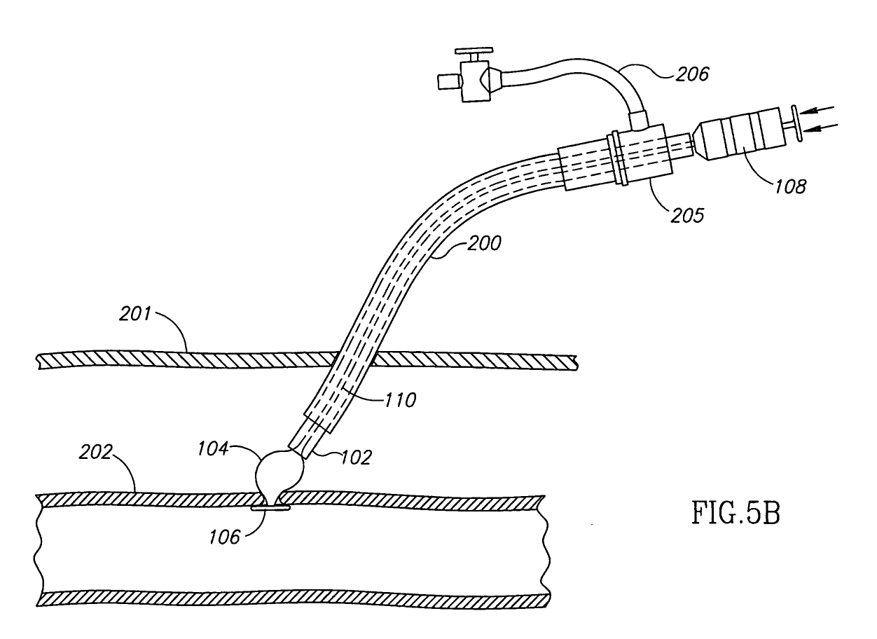

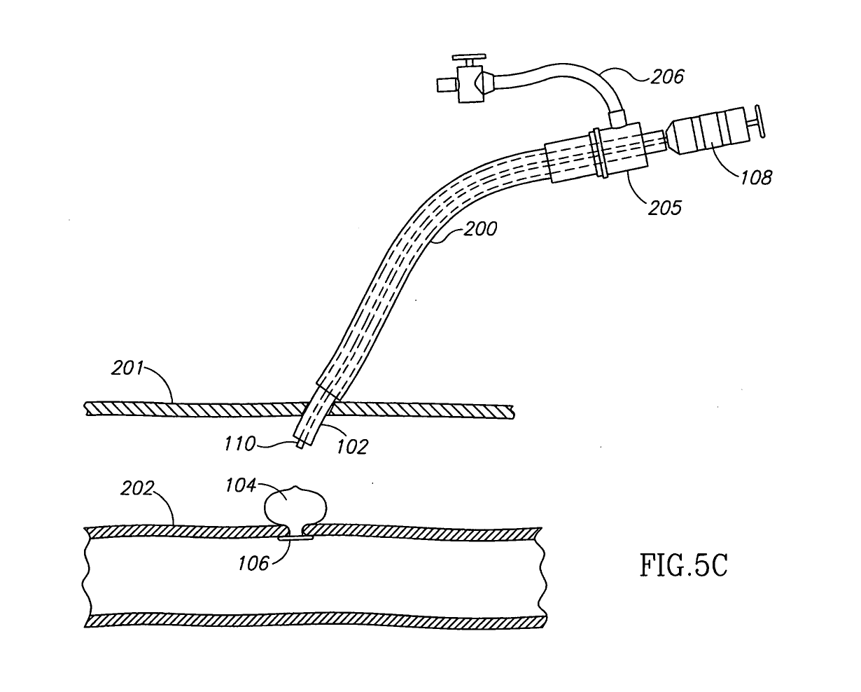

FIGS. 3, 5A, 5B, and 5C are a time sequence, showing how system 100 is used to close up hole 204 and prevent bleeding from blood vessel 202. In FIG. 5A, introducer sheath 200 has been withdrawn from blood vessel 202, although it still remains under skin 201. In particular, sheath 200 has been pulled back until anchor 106 reaches the inner wall of blood vessel 202. Because anchor 106 is now oriented with its long direction perpendicular to the axis of tube 102, anchor 106 does not fit through hole 204, and remains inside blood vessel 202.

Enough force is optionally used, in pulling on sheath 200, so that anchor 106 is firmly pressed against the inner wall of blood vessel 202 at hole 204, reducing or even preventing blood loss through hole 204. Anchor 106 is optionally made wide enough so that it covers the width or almost all of the width of hole 204, but is narrow enough to just fit within sheath 200. The pulling force on sheath 200 is optionally transmitted by friction, for example, or by a clamping mechanism (not shown), through tubes 102 and 110 to balloon 104, and hence to anchor 106. Alternatively, no pulling force, or not enough pulling force, is exerted on anchor 106, or anchor 106 is too narrow to cover hole 204 very well, and some bleeding does occur for a short time, until the balloon is inflated, as presently described.

Anchor 106 need not be a solid shape, but, particularly if the anchor is not being relied upon for covering hole 204, it can be a mesh, for example. Making the anchor in the form of a mesh has the potential advantage that the anchor may tend to be absorbed into the blood vessel wall more quickly, and a piece of the anchor may be less likely to break off into the bloodstream. Optionally, the anchor has other features which may prevent it from breaking off into the bloodstream, for example the anchor optionally has an adhesive on the side that is facing the blood vessel wall, and it is optionally held together with wires. Additionally, the anchor is optionally coated with anti-thrombolytic agents on the side facing the bloodstream, and/or has other features, for example its shape and/or the texture of its surface, which may prevent the anchor from inducing the formation of a thrombosis. Additionally, the anchor optionally has other features which may cause it to be absorbed into the blood vessel wall quickly, particularly if the anchor is not required to hold the balloon in place once it has expanded, because the pressure of surrounding body tissue is sufficient to hold the balloon in place. In this case especially, the anchor is optionally absorbed into the body in much less time than the balloon, due, for example, to a different composition of the anchor, or different physical or chemical surface properties. For example, optionally the anchor is absorbed in one day, or two days, or one week, or two weeks, or any period of time intermediate between these values, or a longer or shorter time than these values.

Balloon 104, which is attached to anchor 106, is located just outside blood vessel 202, adjacent to hole 204. Tube 102 has also been pulled back slightly, relative to balloon 104, uncovering balloon 104 so that it can expand.

In FIG. 5B, syringe 108 is depressed, so that saline solution goes through tube 110 into balloon 104, inflating balloon 104. Because the balloon is surrounded by soft body tissue, for example muscle, inflating the balloon causes the balloon to press against hole 204, as well as against the body tissue surrounding the balloon on its other sides. This pressure against hole 204 will generally substantially prevent bleeding, even in the absence of any pulling force exerted on the balloon and the anchorthroughtubes 102 and 110.

The pressure inside the balloon is optionally less than 0.5 bars, or between 0.5 and 1.0 bars, or between 1.0 and 2.0 bars, or between 2.0 and 3.0 bars, or greater than 3 bars. The pressure difference between the inside and outside of the balloon is optionally less than 0.5 bars, or between 0.5 and 1.0 bars, or between 1.0 and 2.0 bars, or between 2.0 and 3.0 bars, or greater than 3.0 bars. The pressure with which the balloon presses against blood vessel 202 is optionally less than 0.5 bars, or between 0.5 and 1.0 bars, or between 1.0 and 2.0 bars, or between 2.0 and 3.0 bars, or greater than 3.0 bars.

The diameter of balloon 104, when expanded to the desired internal pressure, optionally has a diameter between 2 mm and 5 mm, or between 5 mm and 10 mm, or between 10 mm and 20 mm, or more than 20 mm, or less than 2 mm, depending, for example, on the unexpanded size of the balloon, the elasticity of the balloon, and the compressibility of the tissue surrounding it. In an exemplary embodiment of the invention, the balloon is sized to match a hole type, for example, a puncture using a cardiac catheter or a short incision, for example, 1-3 mm in length. In an exemplary embodiment of the invention, the balloon is sized and pressurized to match certain blood vessels, for example a femoral artery, a carotid artery, a coronary artery or other vessels, for example, of a diameter between 1 and 4 mm, or smaller or larger.

In FIG. 5C, tube 110 is detached from balloon 104, by pulling back on tube 110. Once balloon 104 has expanded, the body tissue surrounding the balloon prevents it from going back along the opening made by introducer sheath 200, which is narrower than the expanded balloon.

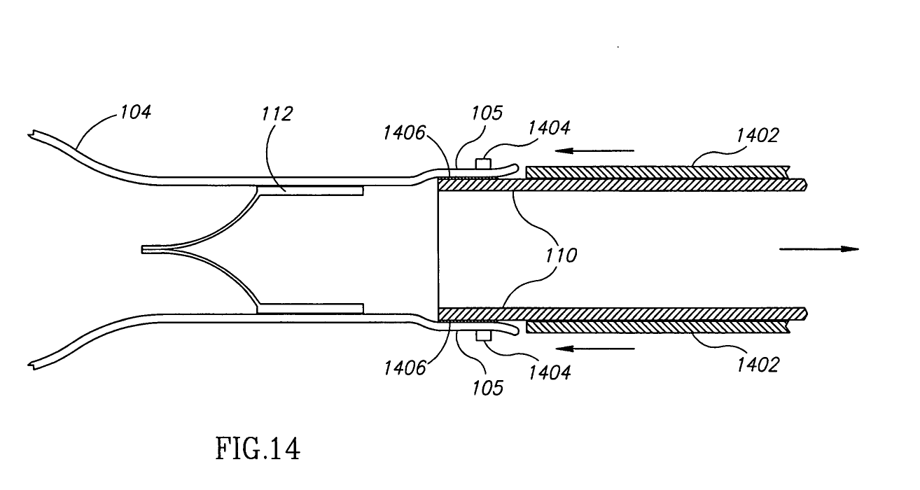

Tube 110 optionally has a location, for example a weakened portion, where it breaks off near balloon 104, proximal to check valve 112, when it is pulled with sufficient force, before tube 110 breaks at a different location, and before the pulling does any damage to balloon 104, anchor 106, or the wall of blood vessel 202, or other body tissue adjacent to the balloon. Check valve 112 thus seals the balloon. Sheath 200, together with tubes 102 and 110 and the rest of system 100, is then withdrawn from the body completely, leaving balloon 104 and anchor 106 in place on blood vessel 202, sealing hole 204. An alternative method of removing tube 110 from balloon 104 is described below, in connection with FIG. 14.

Balloon 104, including check valve 112, and anchor 106, are biodegradable, and eventually disintegrate and are absorbed into the body. As balloon 104 and check valve 112 start to disintegrate, the saline solution may leak out, relieving the pressure in balloon 104, and the pressure that balloon 104 exerts on the wall of blood vessel 202, but by the time that happens, hole 204 is optionally fully healed, or sufficiently healed that there is little danger it will start bleeding again. For example, the balloon is optionally absorbed in one day, or two days, or one week, or two weeks, or any period of time intermediate between these values, or a longer or shorter time than these values. Optionally, the physician can chose between different balloons with different absorption times depending on the size and location of hole 204, the age and medical condition of the patient, and other factors which may influence the desired absorption time.

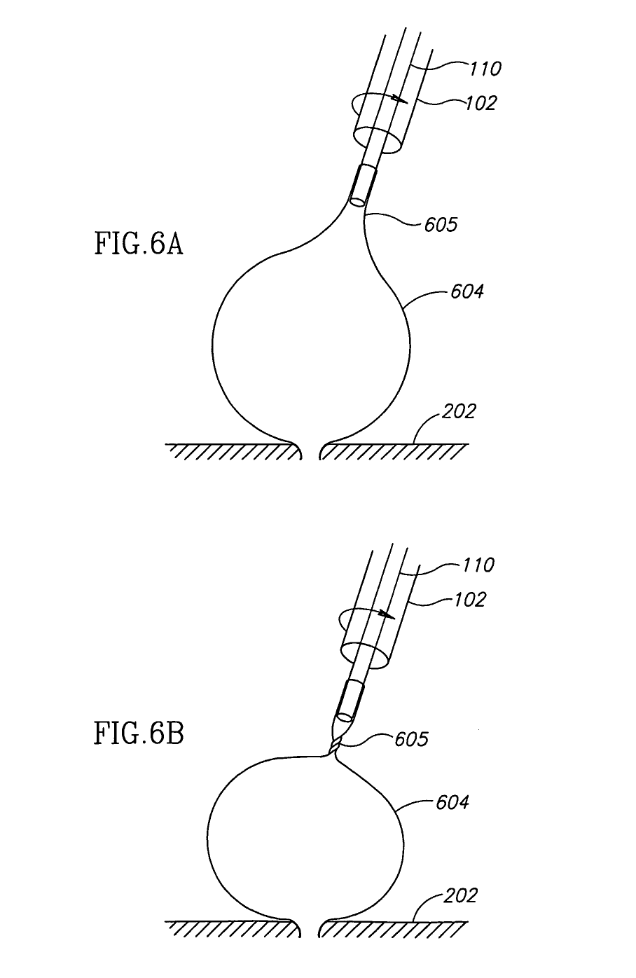

Alternatively, instead of or in addition to using check valve 112 to prevent the saline solution from leaking out of the filled balloon, other means are used to seal the balloon. One such means is shown in FIGS. 6A and 6B. FIG. 6A shows a filled balloon 604, without a check valve, in place in the body, and connected to filling tube 110 at a neck 605. In FIG. 6B, tube 110 is twisted by several full turns, for example by twisting the entire system 100. Balloon 604, because it is surrounded by body tissue, does not turn, but remains stationary while tube 110 is twisted. As a result, neck 605 becomes twisted, with the sides of neck 605 touching each other. Neck 605 is preferably made of or lined with a self-stick material, and when it is twisted, the sides stick together, sealing balloon 604. Pulling on tube 110 then breaks tube 110 just beyond the seal made by neck 605, so that the rest of the hemostasis system and the introducer sheath can be removed from the body, leaving balloon 604 in place.

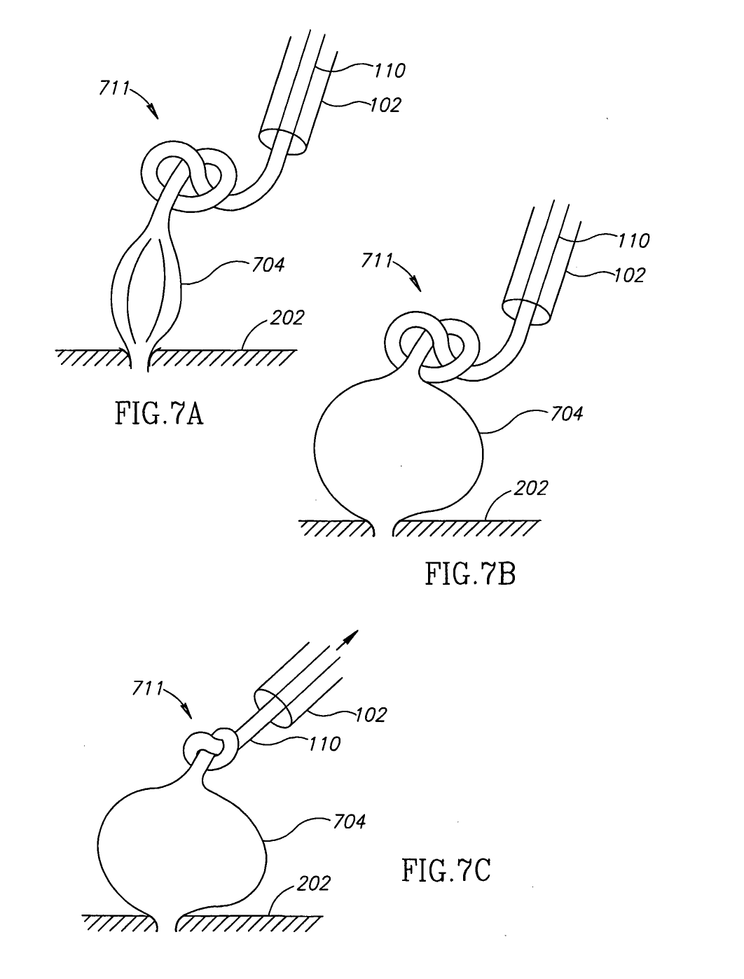

Still another method of sealing a balloon is shown in FIGS. 7A-7C. In FIG. 7A, an uninflated balloon 704, without a check valve, is connected to tube 110, which has been loosely tied in a knot 711, for example, when the hemostasis system was assembled. Because knot 711 is loose, saline solution can flow through it to inflate balloon 704 in FIG. 7B. Once balloon 704 in inflated, tube 110 is pulled, tightening knot 711, as shown in FIG. 7C, and sealing balloon 704. Further pulling on tube 110 optionally causes tube 110 to break off just beyond knot 711, leaving the sealed balloon in place while the rest of the hemostasis system and the introducer sheath are removed from the body.

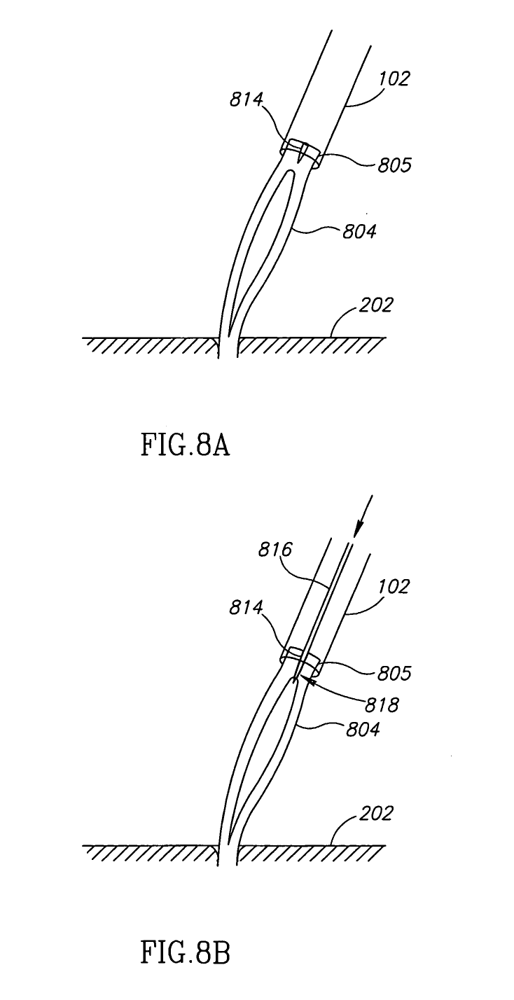

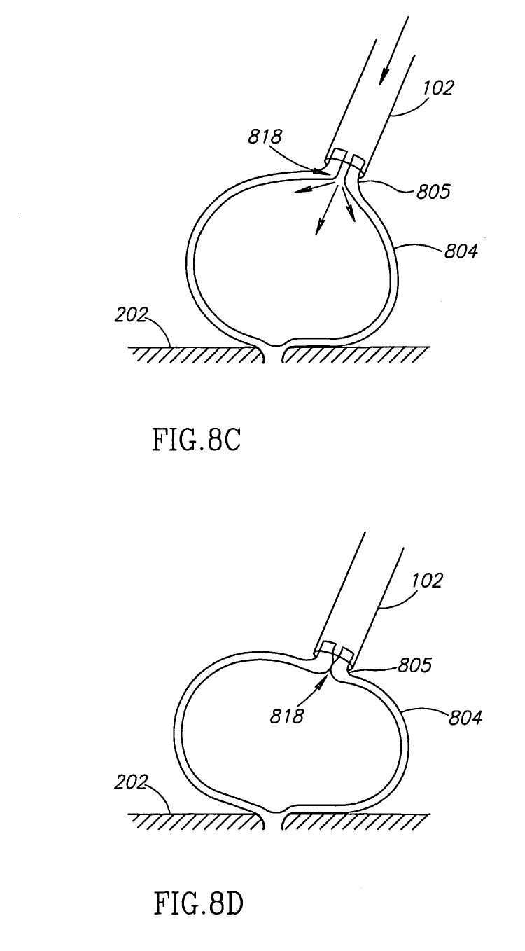

Yet another method of sealing a balloon is shown in FIGS. 8A-8C. FIG. 8A shows a sealed uninflated balloon 804, with a neck 805 that optionally fits snugly into the distal end of tube 102. There is no opening in the balloon, but optionally neck 805 has a channel 814 which goes most of the way through. When the balloon is ready to be inflated, a needle 816 passes through tube 102 and channel 814, as shown in FIG. 8B, making a small puncture 818 in the balloon, at the end of channel 814. Saline solution is then passed into the balloon through tube 102, expanding the balloon, as shown in FIG. 8C. As long as the saline solution flows under pressure into the balloon, it holds open puncture 818. When the balloon is inflated, and the pressure of the saline solution in tube 102 is no longer greater than the pressure inside the balloon, then the saline solution stops flowing into the balloon, and puncture 818 closes up again, as shown in FIG. 8D, sealing itself due to the elasticity of the material.

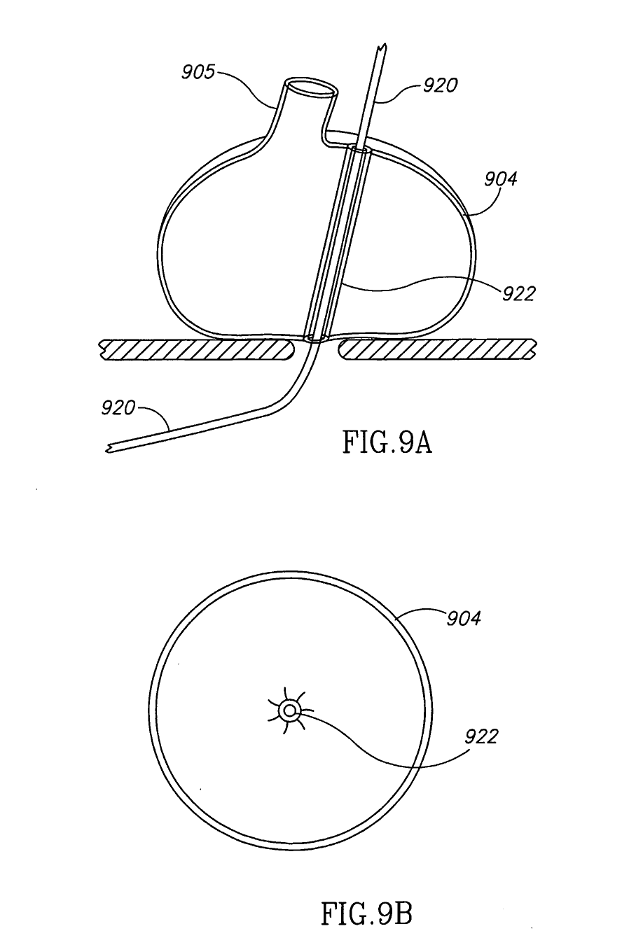

Optionally, instead of using an anchor to position the balloon over the hole in the blood vessel, as in FIGS. 1A-5C, a guide wire is used to position the balloon. The different ways of sealing the balloon shown in FIGS. 6A-8B, as well as the check valve shown in FIG. 1B, can be used with either an anchor or a guide wire. FIG. 9A shows a side view of an inflated balloon 904 with a guide wire 920 running through it. Balloon 904 is topologically a torus, with a channel 922 running through it, for the guide wire to go through. Channel 922 is preferably narrow, for example barely wide enough for the guide wire to go through it, so that little or no blood will leak through channel 922. Balloon 904 also has a neck 905 through which it is inflated. FIG. 9B shows an axial view of balloon 904, with channel 922 seen along its axis. Even in the absence of an anchor, balloon 904 can be expected to be held in place by the surrounding body tissue, which exerts a force on the balloon after it is inflated.

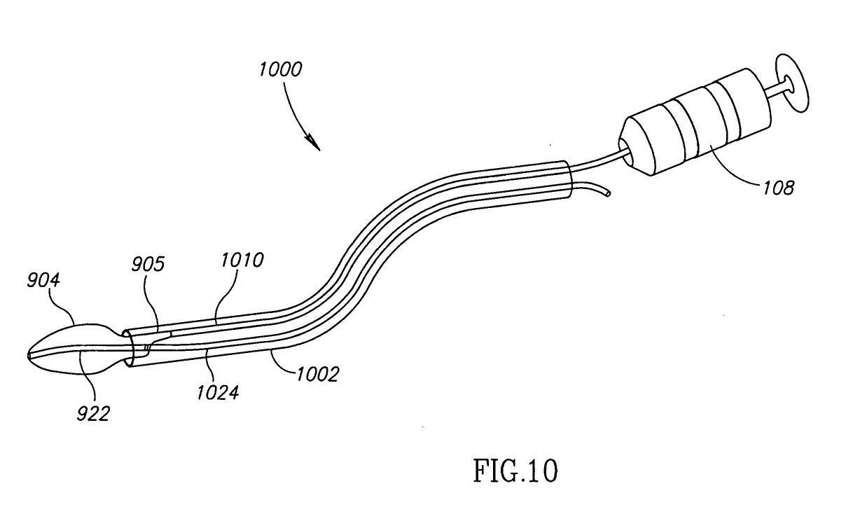

FIG. 10 is a side view of a hemostasis system 1000, similar to system 100, but using a balloon with a guide wire, instead of a balloon with an anchor. Flexible tube 1002 has two tubes running through it side by side, a filling tube 1010, and a guide wire tube 1024. Guide wire tube 1024 is connected to channel 922 in balloon 904, and guide wire 920 runs through channel 922 and tube 1024. The distal end of filling tube 1010 is connected to neck 905 of the balloon. The proximal end of filling tube 1010 is connected to syringe 108, like filling tube 110 in FIG. 1A. Syringe 108 is filled with saline solution which is used to expand the balloon.

Optionally, instead of separate tubes 1024 and 1010 running through tube 1002, tube 1002 is solid except for two bores 1024 and 1010 running through it. Alternatively, one of tubes 1024 or 1010 runs through tube 1002, and the rest of the interior of tube 1002 functions as the other tube, either a filling tube or a guide wire tube. Alternatively, there is no tube 1002. In this case, tubes 1024 and 1010 are optionally tied together in some way along their lengths, for ease in using hemostasis system 1000. However, having tube 1002 has the potential advantage of making hemostatis system stiffer and easier to push into a blood vessel.

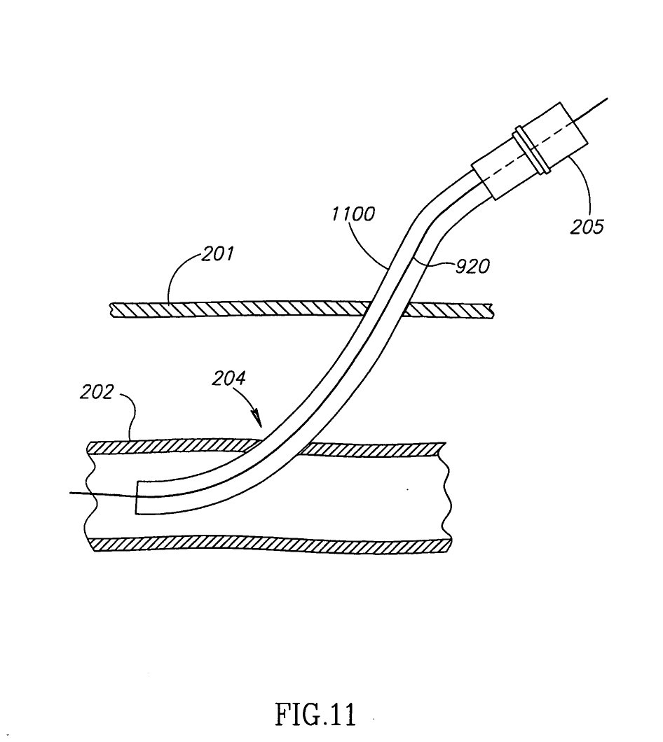

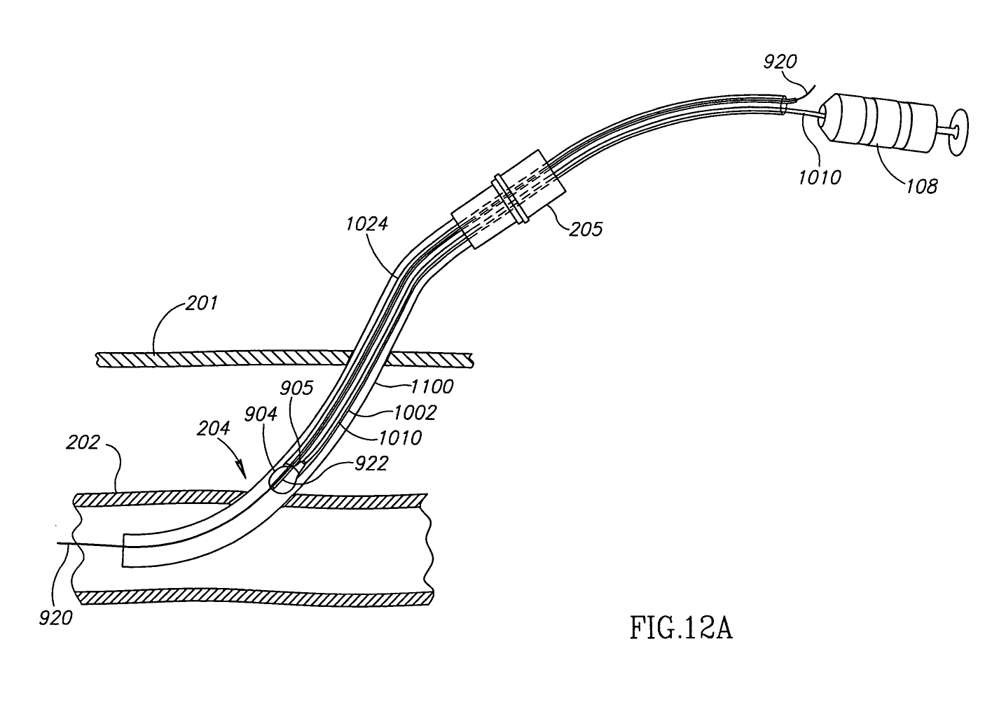

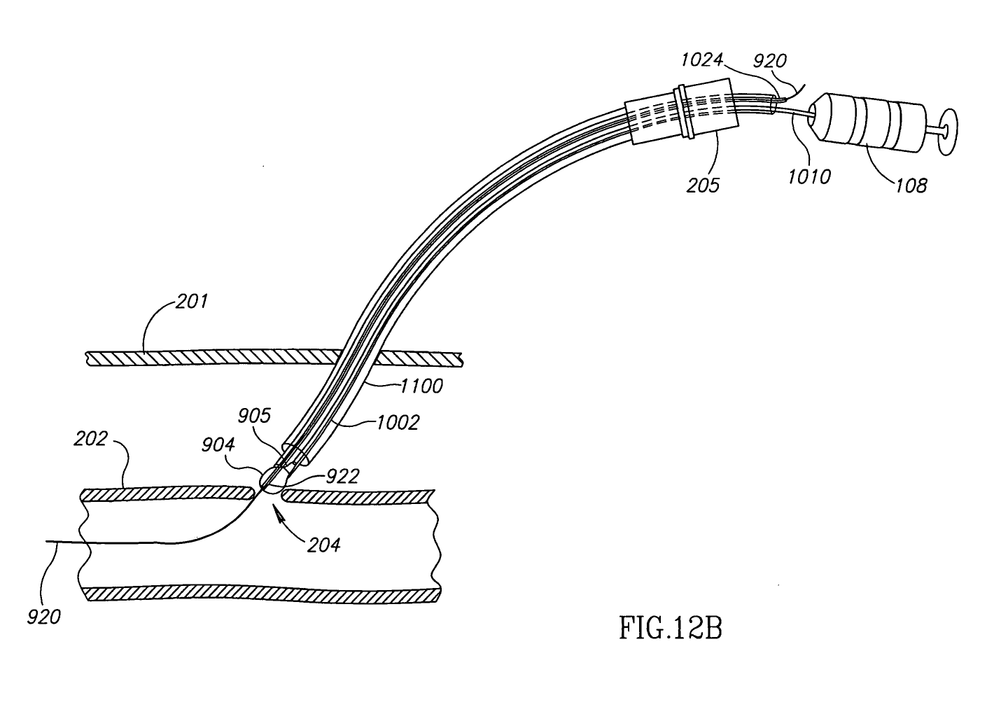

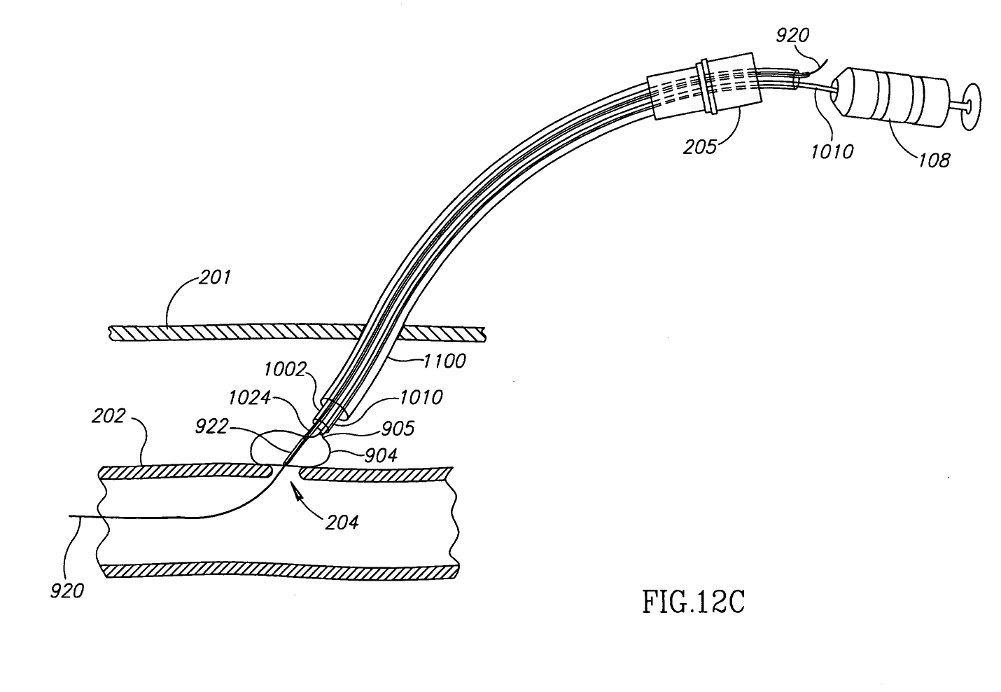

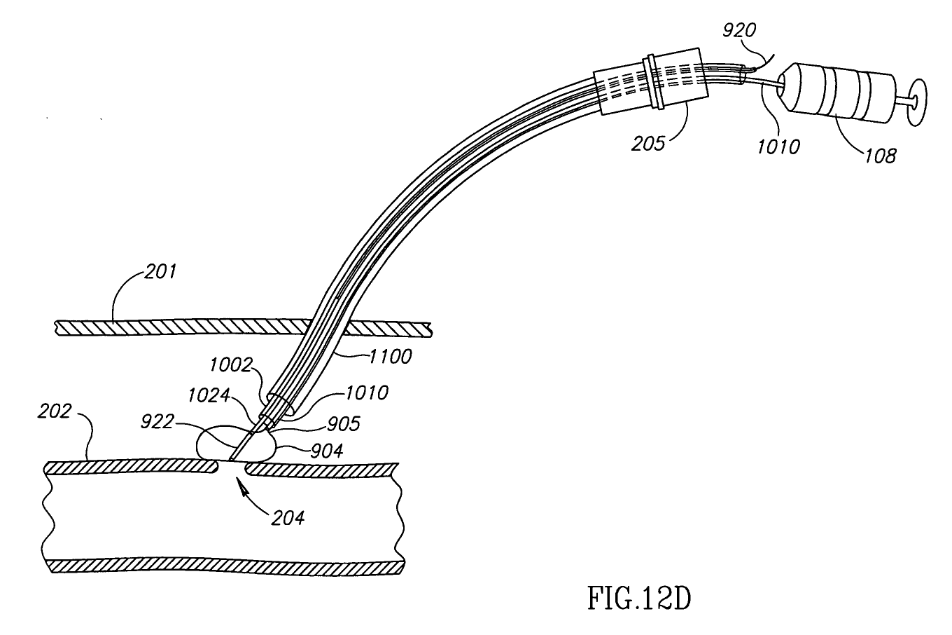

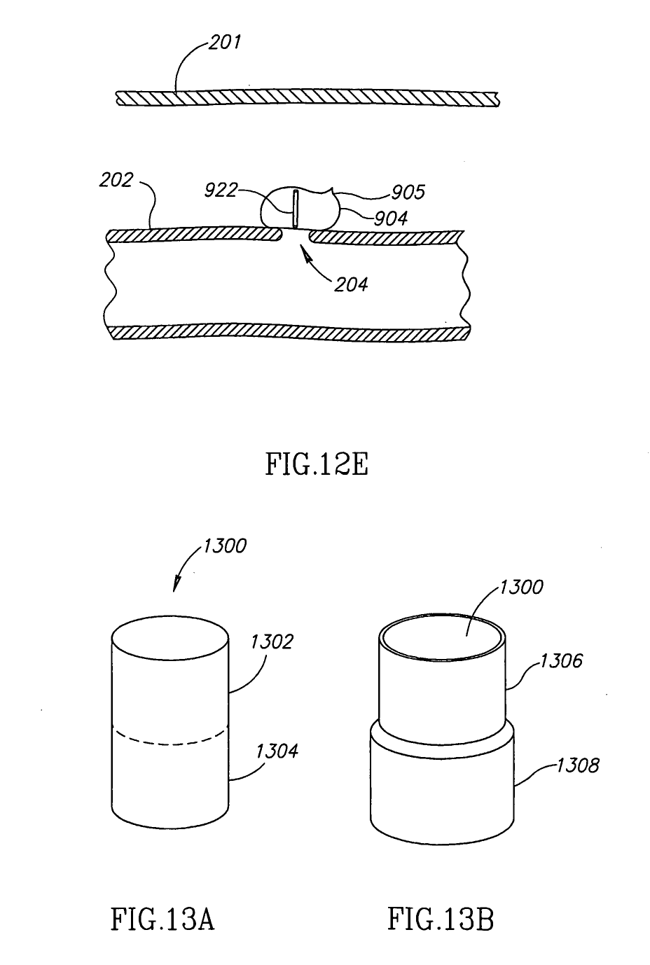

The method of operation of hemostasis system 1000 is shown in FIGS. 11 and 12A-12E. FIG. 11 shows an introducer sheath 1100, similar to introducer sheath 200 in FIG. 2, which is used for introducing a catheter, not shown, into blood vessel 202, through hole 204. Introducer sheath 1100 has a guide wire 920 running through it and into the blood vessel. Optionally, guide wire 920 is also used for guiding the catheter. In FIG. 11, a catheterization procedure has been completed, and the catheter has been removed from the patient's body, but guide wire 920 remains in place.

In FIG. 12A, tube 1002 of hemostasis system 1000 has been pushed into sheath 1100, and guide wire 920 has been threaded through tube 1024 inside tube 1002. Uninflated balloon 904 is attached to the end of tube 1002, and has been pushed through introducer sheath 1100 as far as the outer surface of the wall of blood vessel 202. Some techniques for determining when the balloon is in the correct position are described below. In any case, the balloon is not pushed past the outer wall of blood vessel 202. In FIG. 12B, introducer sheath 1100 has been withdrawn from blood vessel 202, uncovering balloon 904, which now touches blood vessel 202 at the location of hole 204, or is close to blood vessel 202 and just outside it (as shown). Guide wire 920, which runs through balloon 904 remains in place in the blood vessel, keeping balloon 904 centered over hole 204.

In FIG. 12C, the plunger of syringe 108 is depressed, causing saline solution to flow through filling tube 1010, inflating balloon 904. As balloon 904 expands, it presses against the outer wall of blood vessel 202, even if balloon 904 was not quite touching blood vessel 202 before being inflated. The pressure from balloon 904, which also pushes against the body tissue surrounding balloon 904 on its other sides, seals hole 204, substantially preventing blood from leaking out of blood vessel 202. Once balloon 904 is inflated, guide wire 920 is withdrawn from the body, as shown in FIG. 12D. Inflated balloon 904 may be expected to remain in place against hole 204, even without the guide wire, since the surrounding body tissue presses against the balloon, which tends to holds the balloon in place. Optionally, balloon 904 has features which help it remain in place, for example nubs which project into the surrounding tissue, or a rough surface texture, or an adhesive on the surface which sticks to the surrounding tissue (but preferably does not stick to itself when the balloon is folded up before being inflated).

Optionally, the internal pressure of the balloon, and/or the elasticity of the balloon material, closes channel 922 after the guide wire is withdrawn, so that no blood leaks out through channel 922. Alternatively, channel 922 is not completely closed, but is narrow enough that there is no significant leakage of blood.

Finally, filling tube 1010 is detached from balloon 904, optionally after using any of the methods shown in FIGS. 6A-8C for sealing neck 905 of the balloon, or using a check valve in the neck of the balloon to seal it. Optionally, filling tube 1010 is detached from balloon 904 using the same method described below, in FIG. 14, for detaching filling tube 110 from balloon 104. Tubes 1010, 1024, and 1002, and introducer sheath 1100, are then removed from the body, leaving balloon 904, sealed and inflated, in place, as shown in FIG. 12E. Eventually, after hole 204 has healed, balloon 904 disintegrates and is absorbed by the body.

There are several possible methods of determining when the balloon is positioned correctly in FIG. 12A. For example, the introducer sheath optionally has a side lumen, such as side lumen 206 in FIG. 2, which is normally closed to keep blood from leaking out. To determine when the distal end of the introducer sheath reaches the outer wall of the artery, the side port is temporarily opened, and the introducer sheath is gradually withdrawn. Blood will stop coming out of the side lumen when the introducer sheath has been withdrawn past the outer wall of the artery. Alternatively or additionally, the depth of the introducer sheath is monitored when it is put into the blood vessel, so it is known how far past the wall of the blood vessel the end of the introducer sheath is, and the position of the balloon is monitored relative to the end of the introducer sheath. Alternatively or additionally, a contrast medium is injected into the blood vessel, the balloon optionally has a radio-opaque marker, and a fluoroscope is used to indicate how far the balloon is from the blood vessel wall.

Using an anchor, as in FIGS. 5A-5C, has the potential advantage, compared to using a guide wire, that it may be easier to place the balloon outside the wall of the blood vessel, and the placement of the balloon may be more reliable. The anchor method also has the potential advantage of better keeping the balloon in place.

The biodegradable material which the balloon is made out of, as well as the optional anchor and check valve, is optionally a polymer, for example polyglycolide, polycaprolactone, polydioxanone, polylactide and/or copolymers thereof, or poly(lactate-caprolactone). Additionally or alternatively, the biodegradable material is a protein, for example collagen. Additionally or alternatively, the biodegradable material is polysaccharide, polyhyularonic acid, poly L-lactide or poly DL-lactide.

FIGS. 13A-13F show a method of manufacturing a biodegradable leaf valve, suitable for use as check valve 112 shown in FIG. 1B, and FIGS. 13G and 13H show how the valve works. In FIG. 13A, a rod 1300, or a hollow tube, made for example of stainless steel, is plated with a biodegradable material. Optionally, any of the biodegradable materials listed above for the balloon are used to make the valve. Rod 1300, for example, is 1 mm in outer diameter, to produce a valve with an inner diameter of 1 mm. A first portion 1302 of rod 1300 is plated with a thinner layer of the biodegradable material, for example 5 to 10 microns thick in the case of a 1 mm diameter tube, while the a second portion 1304 of rod 1300 is plated with a thicker layer of the biodegradable material, for example 0.1 mm thick. FIG. 13B shows rod 1300 after it has been plated, with a thin plating 1306 covering portion 1302, and a thick plating 1308 covering portion 1304. The plating is then peeled off rod 1300, forming a tube 1310 of the biodegradable material, shown in FIG. 13C, with a thin region 1306 and a thick region 1308. Thin region 1306 is then crimped, for example with tweezers, while being heated, so that it will be somewhat plastic and will not crack. The result is a valve 1312, with a tubular base 1308, and leaves 1306, as shown in FIG. 13D. FIGS. 13E and 13F show valve 1312 from two different side points of view 90 degrees, to make it clear how it is shaped.

Alternatively, instead of plating the biodegradable material on a cylindrical rod and then crimping it, the biodegradable material is plated on a mandrel that is shaped like valve 1312, and is peeled off, so it is not necessary to crimp it to form the valve. Alternatively, valve 1312 is machined, or molded, or manufactured in any other way known to the art. In these cases, leaves 1306 of valve 1312 need not flare out as seen in FIG. 13E, but optionally have a different shape.

FIG. 13F shows how valve 1312 functions when it is in place in neck 105 of balloon 104, with the end of filling tube 110 connected to neck 105. As long as filling tube 110 is filled with material, for example saline solution, under greater pressure than the interior of the balloon, the material flows past leaves 1306, holding the leaves open. Once the balloon is fully inflated, and neck 105 is disconnected from filling tube 110, as shown in FIG. 13G, or even before that when filling tube 110 no longer has a greater pressure inside it than balloon 104, the pressure of the material inside balloon 104 forces leaves 1306 shut, and the balloon remains sealed.

FIG. 14 shows a method of removing balloon 104 from filling tube 110, once balloon 104 has been fully expanded. Neck 105 of balloon 104 has previously been placed over the end of filling tube 110, for example during the assembly of system 100. When it is time to remove the balloon from the filling tube, a pusher tube 1402, which surrounds filling tube 110, is pushed against the end of neck 105, while at the same time filling tube 110 is pulled away from neck 105. Alternatively, a pulling force is applied to filling tube 110, and the inertia of pushing tube 1402 keeps neck 105 from moving with filling tube 110, or a pushing force is applied to pushing tube 1402 and the inertia of filling tube 110 keeps filling tube 110 from moving with neck 105. Preferably, pusher tube 1402 is stiff enough so that it can be pushed, with sufficient force to remove balloon 104, from the proximal end of pusher tube 1402 outside the body. Optionally, pusher tube is placed around filling tube 110 during the assembly of system 100. Alternatively, pusher tube 1402 is only introduced when system 100 is already inside the body.

Optionally, neck 105 is stretched in order to place it around the end of the filling tube 110, and the elastic force of neck 105 holds it on filling tube 110. Alternatively or additionally, a clamp 1404 is place around neck 105 to hold it onto filling tube 110. Alternatively or additionally, a layer of glue 1406 is used between neck 105 and filling tube 110, to hold them together. These or other means known to the art are used to keep neck 105 attached to filling tube 110 firmly enough so that neck 105 will not come off filling tube 110 prematurely, before the balloon is fully inflated, as a result of the pressure in filling tube 110, but neck 105 is not attached so strongly to filling tube 110 that it cannot be removed by pushing on pushing tube 1402 and pulling on filling tube 110.

Optionally, the balloon is folded when it is in the collapsed state, and unfolds when it expands, and optionally stretches as well. Alternatively, the balloon expands entirely by stretching. Since the balloon is optionally only expanded once, the stretching is optionally by plastic deformation (irreversible) or alternatively by elastic deformation (reversible) or by elastic-plastic deformation (partly reversible). Optionally, the balloon increases its diameter by a factor between 2 and 4, or between 4 and 6, and or between 6 and 10, when it is inflated.

Alternatively, instead of inflating the balloon with saline solution and sealing the balloon, the balloon is inflated by filling it with a curable biodegradable material that is biocompatible and absorbable by the body. The material is, for example, a derivative of collagen, fibrin glue, or hydrogel. Optionally, in this case, the balloon is not sealed at all, but the cured material remains in the balloon even without sealing it.

Optionally, particularly if the balloon is sealed by twisting the neck as in FIGS. 6A-6B, or by self-sealing as in FIGS. 8A-8C, the balloon is made of a self-adhesive material. Optionally, in order to prevent self-adhesion of the balloon, the internal surface, or external surface, or both, are coated, at least in part, with a non-sticking material, for example CarboWax 3350 (polyethylene glycol). Coating the external surface may be useful particularly if the balloon is folded initially.

Optionally, the balloon has a non-uniform wall thickness, for example in order to cause the balloon to expand into a non-spherical shape.

The invention has been described in the context of the best mode for carrying it out. It should be understood that not all features shown in the drawings or described in the associated text may be present in an actual device, in accordance with some embodiments of the invention. Furthermore, variations on the method and apparatus shown are included within the scope of the invention, which is limited only by the claims. Also, features of one embodiment may be provided in conjunction with features of a different embodiment of the invention. As used herein, the terms “have”, “include” and “comprise” or their conjugates mean “including but not limited to.”

1. A homeostasis device the device comprising:
(a) a biodegradable balloon adapted to exert pressure on a hole formed in a lumen in the body when the balloon is placed inside the body adjacent to the hole and expanded, the balloon held in place thereafter and further adapted to be absorbed by the body: and
(b) a non-inflatable biodegradable anchor element coupled to the balloon and adapted to remain inside the lumen and hold the balloon in place outside the lumen.

2-5. (canceled)

6. A homeostasis device according to claim 1, wherein the balloon is adapted to exert enough pressure to substantially stop bleeding from the hole, when the lumen is a blood vessel.

7. A homeostasis device according to claim 1, wherein the hole is a catheterization puncture in the blood vessel.

8. A homeostasis device according to claim 7, wherein the blood vessel is an artery.

9. A homeostasis device according to claim 1, wherein said balloon is inflated to a pressure of at most 1 bar.

10. A homeostasis device according to claim 1, wherein said balloon is inflated to a pressure of at most 6 bar.

11. A homeostasis device according to claim 1, wherein said balloon is elastically deformable when it expands.

12. A homeostasis device according to claim 1, wherein said balloon plastically deforms when it expands.

13. A homeostasis device according to claim 1, comprising a channel for a guide wire.

14. A homeostasis device according to claim 1, comprising a sealing mechanism.

15. A homeostasis device according to claim 14, wherein said sealing mechanism comprises a valve.

16. A homeostasis device according to claim 14, wherein said sealing mechanism comprises a self-adhesive channel.

17. A homeostasis device according to claim 14, wherein said sealing mechanism comprises a self-sealing channel.

18-20. (canceled)

21. A homeostasis device according to claim 1, coated on an inside surface thereof with an anti-adhesive material.

22-27. (canceled)

28. A biodegradable check valve adapted to seal an inflatable biodegradable balloon implanted inside the body.

29-33. (canceled)

34. A method of sealing an opening in a hollow structure in the body, the method comprising:
a) positioning an uninflated biodegradable balloon outside the structure, adjacent to the opening;
b) inflating the balloon, causing the balloon to press against the opening, at least partially sealing it;
c) leaving the balloon in place until it degrades and is absorbed by the body; and
d) anchoring the balloon using a non-inflatable biodegradable anchor element attached to said balloon said anchor element positioned inside the structure,
wherein the balloon does not degrade sufficiently to stop pressing against the opening until after the opening seals.

35-46. (canceled)

47. A homeostasis device according to claim 1, wherein at least a portion of the device comprises a biodegradable polymer.

48. A homeostasis device according to claim 1, wherein at/or any other biodegradable material.

49. A homeostasis device according to claim 1, wherein at least a portion of the device comprises a biodegradable material which is neither a protein nor a polymer.

50. A homeostasis device according to claim 1, at least a portion of the device comprises at least one biodegradable materials selected from the group consisting a polysaccharide, a polyhyularonic acid, a poly L-lactide and a poly DL-lactide.
